(19) United States
(12) Patent Application Publication
(10) Pub. No.: US 2026/0124160 A1
(43) Pub. Date: May 7, 2026

(54) PHARMACEUTICAL FORMULATIONS OF CURCUMIN AND/OR RESVERATROL COMPOUNDS

(71) Applicant: Caliway Biopharmaceuticals Co., Ltd., New Taipei City (TW)

(72) Inventor: Yu-Fang LING, New Taipei City (TW)

(21) Appl. No.: 19/381,475

(22) Filed: Nov. 6, 2025

Related U.S. Application Data
(60) Provisional application No. 63/716,970, filed on Nov. 6, 2024.

(51) Int. Cl. A61K 31/12; A61K 9/51; A61K 31/05; A61P 3/04

(52) U.S. Cl. CPC A61K 31/12; A61K 9/5115; A61K 9/5123; A61K 9/5146; A61K 31/05; A61P 3/04

(57) ABSTRACT

A pharmaceutical composition comprising: (a) a resveratrol compound such as resveratrol, a curcumin compound such as curcumin, or a combination thereof; (b) an osmolality adjusting agent, and (c) a non-ionic surfactant, the pharmaceutical composition having an osmolality value ranging from 300±70 mOsm/kg. Also provided herein are therapeutic applications for the pharmaceutical compositions provided herein.

### CROSS REFERENCE TO RELATED APPLICATIONS

This application claims the benefit of the filing date of U.S. Provisional Application No. 63/716,970, filed Nov. 6, 2024, the entire contents of which is incorporated by reference herein.

### BACKGROUND

Curcumin and resveratrol are naturally-occurring compounds with various beneficial effects, including anti-inflammatory and anti-oxidant activities. As such, they are widely used in pharmaceutical compositions to benefit treatment of various diseases and disorders.

Subcutaneous (SC) administration of pharmaceutical formulations is widely used to effectively deliver different drugs to human patients. SC offer highs bioavailability and rapid onset of action and is considered near-ideal pharmacokinetically, less painful, and less prone to infections. Nonetheless, SC injections can involve a delayed response due to absorption at injection site and can cause injection site reactions, including pain that may lead to non-adherence. Injection site pain (ISP) is a subjective side effect that can impact treatment experience and patient adherence. It can be caused by various factors, such as needle features, volume, site and speed of injection, as well as features associated with the formulation (e.g., osmolality, viscosity, and pH). St Clair-Jones et al., Rheumatology and Therapy, vol. 7(4):741-757 (2020).

### SUMMARY

The present disclosure is based, at least in part, on the development of compositions comprising curcumin, resveratrol, or a combination thereof with suitable osmolality values. Such compositions are expected to improve patient experience when delivered by subcutaneous injection (e.g., less pain at the injection site, less edema at the injection site, less inflammatory response at the injection site and/or no or slight subcutaneous hemorrhage).

Accordingly, the present disclosure provides, in some aspects, a pharmaceutical composition, comprising a biologically active agent, a non-ionic surfactant hydrophilic-lipophilic balance (HLB) value ≥10, and an osmolality adjusting agent. The biologically active agent may comprise a curcumin compound (e.g., curcumin), a resveratrol compound (e.g., resveratrol), or a combination thereof. The pharmaceutical composition may have an osmolality value ranging from 300±70 mOsm/kg (e.g., 300±30 mOsm/kg). In some instances, the pharmaceutical composition has a pH value ranging from 3 to 8.5, for example, from 4 to 8.2, or from 4.5 to 7.8. In some examples, the pharmaceutical composition has a pH value ranging from 5.0 to 6.0, for example, 5.6.

In some embodiments, the pharmaceutical composition may comprise curcumin at a concentration ranging from 0.04% to 5% by weight. In some examples, the concentration of curcumin in the composition ranges from 0.1% to 2.5% by weight. In one specific example, the concentration of curcumin in the composition can be about 0.4% by weight. In such a curcumin-containing pharmaceutical composition, the pH of the composition may range from about 5.5 to about 8.5.

Alternatively or in addition, the pharmaceutical composition may comprise or further comprise resveratrol at a concentration of 0.04% to 20% by weight. In some examples, the concentration of resveratrol in the composition ranges from 0.05% to 5% by weight. In some specific examples, the concentration of resveratrol in the composition ranges from 0.05% to 2.5% by weight. When the pharmaceutical composition comprises resveratrol, its pH value may range from about 4.5 to about 8.5.

In some embodiments, the pharmaceutical composition provided herein comprises one or more non-ionic surfactants. Examples include a PEGylated oils, polyoxyethylene, polyoxyethylene castor oil derivatives, polyoxy ethylene, polyoxyl castor oil, polyoxyl hydrogenated castor oil, or a combination thereof. In some examples, the non-ionic surfactant comprises polyoxyl-35 castor oil, polyoxyl 40 hydrogenated castor oil, polyoxyl 15 hydroxystearate. or a combination thereof. Alternatively or in addition, the composition comprises the non-ionic surfactant at a concentration of 5% to 99% by weight (e.g., 5% to 95%, 5% to 90%, 5% to 85%, 5% to 80%, or 5% to 75%). In some instances, the composition is free of water.

In some embodiments, the osmolality adjusting agent in the composition may comprise an organic agent capable of adjusting osmolality values. Examples include dextrose, mannitol, sorbitol, glycerin, and/or propylene glycol. Alternatively or in addition, the osmolality adjusting agent can comprise an inorganic compound, for example, sodium chloride, potassium chloride, dibasic potassium phosphate, sodium hydrogen phosphate, sodium citrate dihydrate, and/or sodium sulfite. In some examples, the osmolality adjusting agent may comprise sodium chloride at a concentration of 0.05% to 0.9% by weight.

In some embodiments, the composition has a weight ratio of the biologically active agent to the non-ionic surfactant ranging from 1:5 to 1:500. In some instances, the non-ionic surfactant and the biologically active agent form a plurality of nanoparticles. In some examples, the nanoparticles may have an average particle size of up to 200 nm and/or a polydispersity index (PDI) value of up to 0.4. In some examples, the nanoparticles may have an average particle size of up to 50 nm and/or a polydispersity index (PDI) value of up to 0.4.

In some examples, the pharmaceutical composition provided herein consists essentially of curcumin, polyoxyl-35 castor oil, osmolality adjusting agent (e.g., sodium chloride), and water. In other examples, the pharmaceutical composition provided herein consists essentially of resveratrol, polyoxyl-35 castor oil, osmolality adjusting agent (e.g., sodium chloride), and water. In yet other examples, the pharmaceutical composition provided herein consists essentially of curcumin, resveratrol, polyoxyl-35 castor oil, osmolality adjusting agent (e.g., sodium chloride), and water. Specific examples are provided in Examples below, each of which is within the scope of the present disclosure.

In another aspect, the present disclosure provides a method for reducing fat and/or adipocytes, thereby benefiting treatment of a disease associated with such. The method may comprise: administering to a subject in need thereof an effective amount of any of the pharmaceutical compositions provided herein. In other aspect, the present disclosure provides a method for alleviating a condition caused by lipoma in a subject. In some examples, the pharmaceutical composition is administered via subcutaneous injection. Exemplary target diseases and disorders include Dercum's disease (Adiposis dolorosa), Gardner syndrome, Familial multiple lipomatosis (Hereditary multiple lipomatosis), Madelung's disease (multiple symmetric lipomatosis), painful lipomas, angiolipomas, lipomatosis, fat disorder, subcutaneous adipose tissue diseases, lipedema, skin disease, a lipid metabolism disorder, a metabolic disease, rare adipose disorders (RADs), Panniculitis, Weber-Christian disease, Progressive lipodystrophy or other diseases/symptoms related to fatty lesions. In some instances, the composition provided herein can be used to reduce local fat (e.g., subcutaneous fat, visceral fat, or fat in respiratory tract) and/or reduce body weight.

Also within the scope of the present disclosure are pharmaceutical compositions for use in achieving the therapeutic benefit disclosed herein, e.g., alleviating any of the target diseases noted herein, as well as use of the pharmaceutical compositions for manufacturing medicaments for use in treating the target diseases.

The details of one or more embodiments of the invention are set forth in the description below. Other features or advantages of the present invention will be apparent from the following drawings and detailed description of several embodiments, and also from the appended claims.

### BRIEF DESCRIPTION OF THE DRAWINGS

The following drawings form part of the present specification and are included to further demonstrate certain aspects of the present disclosure, which can be better understood by reference to the drawing in combination with the detailed description of specific embodiments presented herein.

FIGS. 1A-1C include diagrams showing the reduction of local fat by exemplary compositions as indicated in the diet-induced obesity (DIO) rat model. FIG. 1A: fat volume at the injection site. FIG. 1B: change of fat volume at the injection site. FIG. 1C: body weight.

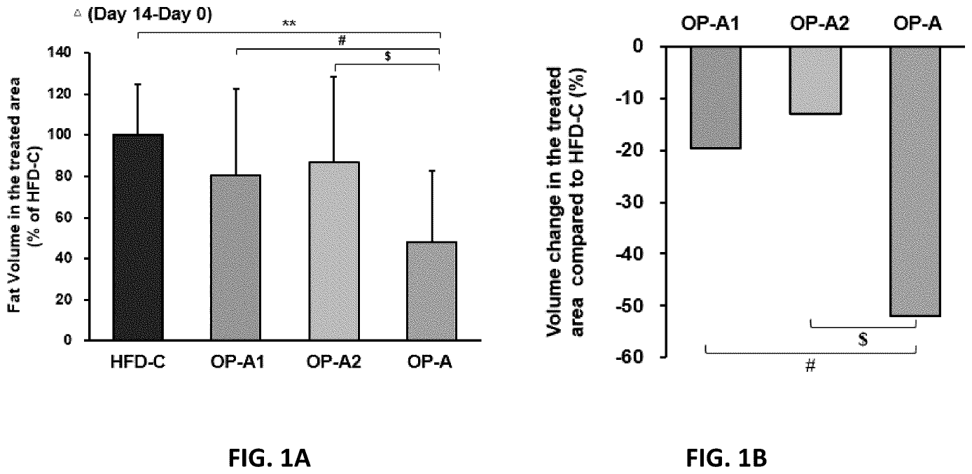

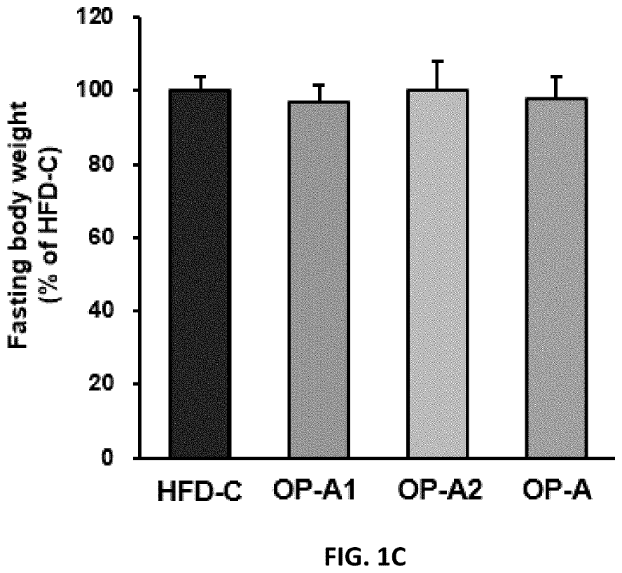

### DETAILED DESCRIPTION

The present disclosure provides a pharmaceutical composition comprising of a resveratrol compound (e.g., resveratrol), a curcumin compound (e.g., curcumin), or a combination thereof, an osmolality adjusting agent, and one or more non-ionic surfactant such as those having a hydrophilic-lipophilic balance (HLB) value equal to or greater than 10. The pharmaceutical composition provided herein has an osmolality value ranging from 300±30 mOsm/kg. In some instances, the pharmaceutical composition has a pH ranging from 3 to 8.5, e.g., 4 to 8.2, 4.5 to 7.8, or 5 to 6 (e.g., 5.6).

Resveratrol (3,4′,5-trihydroxystilbene), a phytoalexin that can mimic the effects of caloric restriction and exert anti-inflammatory and anti-oxidative effects, is reported to benefit treatment of various diseases, including cancer, neurological disorders, cardiovascular diseases, diabetes, non-alcoholic fatty liver disease (NAFLD), and obesity. Despite its attractiveness as a drug candidate, most human trials report poor solubility and bioavailability as a major obstacle. See, e.g., Berman et al., NPJ Precision Oncology, 2017, 35(2017) and Salehi et al., Biomedicines, vol. 6(3): 91, 2018.

Curcumin (1,7-bis-(4-hydroxy-3-methoxyphenyl)-hepta-1,6-diene-3,5-dione), is a polyphenol and has been studied in various disease models, for example, cancer, cardiovascular disease, arthritis, and gastric inflammation. Gupta et al., The AAPS journal vol. 15.1 (2013): 195-218. Similar to resveratrol, poor stability and poor bioavailability are the major hurdles associated with development of curcumin as a therapeutic agent. See, e.g., Lopresti et al., Advances in Nutrition, 2018, 9(1): 41-50.

The pharmaceutical compositions provided herein are expected to possess various advantageous features, for example, to minimize injection site pain, edema, inflammatory response, and/or subcutaneous hemorrhage.

### I. Pharmaceutical Compositions

The pharmaceutical compositions provided herein comprise a biologically active agent, which may comprise a resveratrol compound (e.g., resveratrol), a curcumin compound (e.g., curcumin), or a combination thereof, one or more non-ionic surfactants, and an osmolality adjusting agent.

In some embodiments, the pharmaceutical compositions provided here are formulated for injection, for example, for subcutaneous injection. The pharmaceutical compositions as disclosed here have a suitable osmolality value, for example, ranging from 300±30 mOsm/kg. In some instances, the pharmaceutical composition has a suitable pH value, for example, ranging from 3 to 8.5. In some embodiments, the pharmaceutical composition comprises a curcumin compound such as curcumin and its pH value may range from about 5.5 to about 8.5. In other embodiments, the pharmaceutical composition comprises a resveratrol compound such as resveratrol and its pH value may range from about 4.5 to about 8.5.

### A. Biologically Active Agent

The biologically active agent in any of the pharmaceutical compositions provided herein comprises a resveratrol compound such as resveratrol, a curcumin compound such as curcumin, or a combination thereof.

### (i) Resveratrol Compounds

In some embodiments, the pharmaceutical compositions provided herein comprise one or more resveratrol compounds. As used herein, a resveratrol compound refers to a compound having a core structure of:

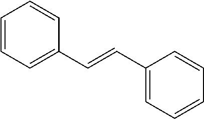 [SMILES: C(=C\c1ccccc1)/c1ccccc1]

with suitable substitutions at one or more positions, for example, on either or both of the ring structures. Suitable substitutions include halogen, thiol, hydroxyl, C₁₋₄ alkyl, C₂₋₄ alkenyl, OR₅, COOR₆, or —OC(═O)R₇, wherein each of R₅, R₆, and R₇ are independently H, or C₁₋₄ alkyl (e.g., methyl). Exemplary C₁₋₄ alkyl groups include, but are not limited to, methyl, ethyl, n-propyl, isopropyl, n-butyl, sec-butyl, isobutyl, tert-butyl. Exemplary C₂₋₄alkenyl groups include, but are not limited to, ethenyl, propenyl, butenyl; alkynyl; aralkyl; halogenated alkyl; heteroalkyl; aryl; heterocyclyl; cycloalkyl; cycloalkenyl; cycloalkynyl.

As used herein, the term “alkyl” refers to a linear, saturated, acyclic, monovalent hydrocarbon radical or branched, saturated, acyclic, monovalent hydrocarbon radical, having from one to three carbon atoms attached to the rest of the molecule by a single bond, e.g., methyl, ethyl, n-propyl, or 1-methylethyl (iso-propyl). An optionally substituted alkyl radical is an alkyl radical that is optionally substituted, valence permitting, by one, two, three, four, or five substituents independently selected from the group consisting of halo, cyano, nitro, oxo, hydroxyl, thio, or amino.

As used herein, the term “alkenyl” refers to a linear, acyclic, monovalent hydrocarbon radical or branched, acyclic, monovalent hydrocarbon radical, containing a carbon-carbon double bond, having two or three carbon atoms attached to the rest of the molecule by a single bond, e.g., ethenyl, or propenyl. An optionally substituted alkenyl radical is an alkenyl radical that is optionally substituted, valence permitting, by one, two, or three substituents independently selected from the group consisting of: halo, cyano, nitro, hydroxyl, thio, or amino. As described herein, an alkene may be a Z or E alkene. An alkene shown as a Z alkene include the E isomer of the alkene.

“Amino” refers to a radical of the formula —NRbRc where Rb and Rc are each hydrogen, or an alkyl radical as defined above containing one to three carbon atoms. The alkyl part of the optionally substituted amino radical is optionally substituted as defined above for an alkyl radical.

“Thiol” refers to a radical of the formula —SRd where Rd is a hydrogen or an alkyl radical as defined above containing one to three carbon atoms. The alkyl part of the optionally substituted thiol radical is optionally substituted as defined above for an alkyl radical.

In some examples, the resveratrol compound may be resveratrol, having the structure of

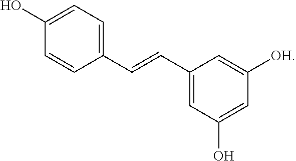 [SMILES: Oc1ccc(/C=C/c2cc(O)cc(O)c2)cc1]

In other examples, the resveratrol compound may be a resveratrol derivative, for example, hydroxylated resveratrol (e.g., oxyresveratrol), methoxylated resveratrol, glycosylated resveratrol, or oligomers of resveratrol.

Compounds described herein can comprise one or more asymmetric centers, and thus can exist in various isomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Tnterscience, New York, 1981); Wilen et al., Tetrahedron 33:2725 (1977); Eliel, Stereochemistiy of Carbon Compounds (McGraw-Hill, NY, 1962); and Wilen, Tables of Resolving Agents and Optical Resolutions p. 268 (E. L. Eliel, Ed., Univ. of Notre Dame Press, Notre Dame, IN 1972). The disclosure additionally encompasses compounds described herein as individual isomers substantially free of other isomers, and alternatively, as mixtures of various isomers.

### (ii) Curcumin Compounds

In some embodiments, the pharmaceutical compositions provided herein comprise one or more curcumin compounds, either taken alone or in combination with one or more resveratrol compounds as also provided herein. As used herein, a curcumin compound refers to a compound having a core structure of:

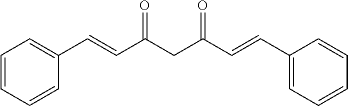 [SMILES: O=C(/C=C/c1ccccc1)CC(=O)/C=C/c1ccccc1]

with suitable substitutions at one or more positions, for example, on either or both of the ring structures. Suitable substitutions include halogen, thiol, hydroxyl, C₁₋₄ alkyl, C₂₋₄ alkenyl, OR₅, COOR₆, or —OC(═O)R₇, wherein each of R₅, R₆, and R₇ are independently H, or C₁₋₄ alkyl (e.g., methyl). Exemplary C₁₋₄ alkyl groups include, but are not limited to, methyl, ethyl, n-propyl, isopropyl, n-butyl, sec-butyl, isobutyl, tert-butyl. Exemplary C₂₋₄alkenyl groups include, but are not limited to, ethenyl, propenyl, butenyl; alkynyl; aralkyl; halogenated alkyl; heteroalkyl; aryl; heterocyclyl; cycloalkyl; cycloalkenyl; cycloalkynyl.

In some examples, the resveratrol compound may be curcumin, having the structure of

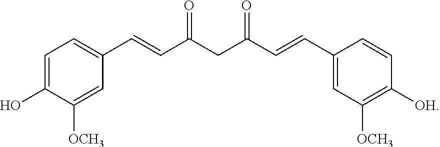 [SMILES: COc1cc(/C=C/C(=O)CC(=O)/C=C/c2ccc(O)c(OC)c2)ccc1O]

In other examples, the curcumin compound may be a curcumin derivative, for example, hydroxylated curcumin, methoxylated curcumin, glycosylated curcumin, demethoxylated curcumin (e.g., demethoxycurcumin and bisdemethoxycurcumin) or oligomers of curcumin. In some instances, curcumin derivatives can be produced by chemical reaction between aryl-aldehydes and acetylacetone.
(iii) Osmolality Adjusting Agents

The pharmaceutical compositions provided herein comprise one or more osmolality adjusting agents. “Osmolality,” which relates to the tonicity of a formulation, refers to the colligative property that depends on the number of dissolved particles in a solution. It is a measurement of the concentration relative to the mass of the solvent, i.e., the number of solute particles in 1 kg of solvent. The common unit of osmolality is mOsm/kg (milliosmoles per kilogram of the solvent such as water).

As used herein, an osmolality adjusting agent refers to a compound that can modulate the osmolality value in a solution. In some embodiments, the osmolality agent contained in the pharmaceutical compositions provided herein can comprise an organic agent capable of modulating osmolality of the composition. Examples include, but are not limited to, dextrose, mannitol, sorbitol, glycerin, propylene glycol (PG), or a combination thereof. Alternatively or in addition, the osmolality adjusting agent provided herein may comprise an inorganic compound, e.g., an ionic salt. Examples include sodium chloride (NaCl), potassium chloride (KCl), dibasic potassium phosphate (K₂HPO₄), sodium hydrogen phosphate (Na₂HPO₄), sodium citrate dihydrate, or sodium sulfite. In some instances, the pharmaceutical composition provided herein may comprise a combination of organic and inorganic osmolality adjusting agents. In one specific example, the pharmaceutical composition comprises sodium chloride as an osmolality adjusting agent.

Any of the osmolality adjusting agents provided herein assist with modulating the osmolality value of the pharmaceutical composition comprising such to 300±70 mOsm/kg (e.g., 300±30 mOsm/kg). In some instances, the pharmaceutical composition may have an osmolality value of 250-350 mOsm/kg. In other instances, the pharmaceutical composition may have an osmolality value of 270-330 mOsm/kg. For example, the pharmaceutical composition may have an osmolality value of 230-300 mOsm/kg (e.g., 230-270 mOsm/kg or 270-300 mOsm/kg). In other examples, the pharmaceutical composition may have an osmolality value of 300-370 mOsm/kg (e.g., 300-330 mOsm/kg or 330-370 mOsm/kg).

### (iv) Non-Ionic Surfactants

The pharmaceutical composition disclosed herein may comprise one or more non-ionic surfactants. In some instances, the non-ionic surfactants may form nanoparticles with the biologically active agent as disclosed herein. In some examples, the nanoparticles may have the effect of encapsulating the biologically active agent such as a curcumin compound, a resveratrol compound or a combination thereof.

Non-ionic surfactants used in any of the pharmaceutical compositions disclosed herein preferably have a hydrophilic-lipophilic balance (HLB) value greater than 5, e.g., ≥10. The non-ionic surfactant is used in the present technology in a ratio as set forth above.

In some embodiments, the non-ionic surfactant comprises a polyoxyl castor oil. Examples include is polyoxyl 5 castor oil, polyoxyl 9 castor oil, polyoxyl 15 castor oil, polyoxyl 35 castor oil, polyoxyl 40 castor oil, polyoxyl 60 castor oil, polyosyl 100 castor oil, or polyoxyl 200 castor oil. In some embodiments, the non-ionic surfactant may comprise or further comprise a hydrogenated polyoxyl castor oil. Examples include polyoxyl 40 hydrogenated castor, polyoxyl 60 hydrogenated castor oil, polyoxyl 100 hydrogenated castor oil, or polyoxyl 200 hydrogenated castor oil. See also KK Singh. Polyoxyethylene Castor Oil Derivatives. 2009; 542-549, the relevant disclosures of which are incorporated by reference for the subject matter and purpose referenced herein. Alternatively or in addition, the non-ionic surfactant comprises or further comprises polyoxyl hydroxystearate, for example, polyoxyl 15 hydroxystearate.

Exemplary non-ionic surfactants include, but are not limited to, polysorbate 80 (Tween® 80), polyoxyl 15 hydroxystearate (Solutol® HS-15), polyoxyethylene castor oil derivatives (e.g., polyoxyl 35 castor oil (Kolliphor® ELP), polyoxyl 40 hydrogenated castor oil (Koliphor® RH40) and polyoxyl 60 hydrogenated castor oil (Cremophor® RH60), Polyoxyethylene (12) glyceryl laurate (UNIGLY ML-212), Polyoxyl 20 Stearate (Myrj™ S20), Polyoxyl 40 Stearate (Myrj™ S40), Polyoxyl 12 Cetostearyl Ether (Kolliphor® CS 12) and Polyoxyl 20 Cetostearyl Ether (Kolliphor® CS 20).

In some instances, the non-ionic surfactant may be a polyoxyethylene molecule, also known as a PEGylated excipient. In some examples, the polyoxyethylene molecule is a PEG castor oil molecule, which can be obtained by reacting varying amounts of ethylene oxide with either castor oil or hydrogenated castor oil. Examples include PEG-35 castor oil and PEG-40 hydrogenerated Castor Oil. In other examples, the polyoxyethylene molecule is a PEG ester, which can be obtained by reacting a polyethylene glycol with a fatty acid. Examples include PEG-40 Stearate and PEG-15 Hydroxystearate. In some instances, the ester may be a sorbitan fatty acid ester (e.g., PEG-20 sorbitan monooleate, PEG-40 sorbitan monooleate, PEG-60 sorbitan monooleate, PEG-80 sorbitan monooleate, PEG-20 sorbitan isostearate, PEG-30 sorbitan tetraoleate, PEG-40, -60 sorbitan tetraoleate, PEG-40 sorbitan diisostearate, or PEG-60 sorbitan tetrastearate), an alkyl glyceride (e.g., PEG-8 Caprylic/capric glycerides, PEG-32 hydrogenated palm glycerides, or PEG-32 Lauroyl glycerides), or an alkyl ether (e.g., PEG-6 cetostearyl ether, PEG-12 cetostearyl ether, PEG-20 cetostearyl ether, PEG-10 cetyl ether, PEG-20 cetyl ether, PEG-4 lauryl ether, PEG-23 lauryl ether, PEG-2 oleyl ether, PEG-10 oleyl ether, PEG-20 oleyl ether, PEG-2 stearyl ether, PEG-10 stearyl ether, PEG-21 stearyl ether, or PEG-100 stearyl ether). In yet other instances, the ester may be a laurate (e.g., PEG-2 laurate, PEG-4 laurate, PEG-6 laurate, PEG-8 laurate. PEG-9-14 laurate, PEG-20 laurate, PEG-32-150 laurate, or PEG-12 glyceryl laurate), a dilaurate (e.g., PEG-2 dilaurate, PEG-4 dilaurate, or PEG-6-150 dilaurate), or a stearate (e.g., PEG-2 stearate, PEG-3 stearate, PEG-4 stearate, PEG-4 isostearate, PEG-5-7 stearate, PEG-6-8 isostearate, PEG-8 stearate, PEG-9 stearate, PEG-10 stearate, PEG-10-isostearate, PEG-12 isostearate, PEG-12-18 stearate, PEG-20 stearate, PEG-23-45 stearate, PEG-40 stearate, PEG-50 stearate, PEG-100 stearate, PEG-75-150 stearate, or PEG-6 and PEG-32 palmitostearate), a glyceryl stearate (e.g., Glyceryl stearate/PEG-40 stearate, Glyceryl stearate/PEG-100 stearate, PEG-120 glyceryl stearate, PEG-20 methyl glucose sesquistearate, or PEG-25 propylene glycol stearate), a distearate (e.g., PEG-2 distearate, PEG-3-120 distearate, PEG-150 distearate, or PEG-175 distearate), a hydroxystarate (e.g., PEG-15 hydroxystearates).

In some instances, the non-ionic surfactant may be a castor oil molecule (e.g., PEG-35 castor oil or PEG-40 castor oil) or a hydrogenated castor oil (e.g., PEG-40 hydrogenated castor oil, PEG-54 hydrogenated castor oil, or PEG-60 hydrogenated castor oil).

Additional examples of non-ionic surfactants include PEG-15 cocamine, Vitamin E polyethylene glycol succinate, PEG-75 lanolin, and PEG-120 methyl glucose dioleate.

### (v) Nanoparticles and Preparations Thereof

In some embodiments, the pharmaceutical compositions disclosed herein may comprise nanoparticles formed by one or more non-ionic surfactants as disclosed herein and the resveratrol compound such as resveratrol, the curcumin compound such as curcumin, or both.

In some instances, the pharmaceutical composition provided herein may comprise nanoparticles such as micelles formed by a non-ionic surfactant as those provided herein and a resveratrol compound such as resveratrol. In other instances, the pharmaceutical composition provided herein may comprise nanoparticles such as micelles formed by a non-ionic surfactant as those provided herein and a curcumin compound such as curcumin. In yet other instances, the pharmaceutical composition provided herein may comprise nanoparticles such as micelles formed by a non-ionic surfactant as those provided herein, a resveratrol compound such as resveratrol, and a curcumin compound such as curcumin. For example, the pharmaceutical composition may comprise one population of nanoparticles such as micelles comprising the non-ionic surfactant and both of the resveratrol compound and the curcumin compound. In other examples, the pharmaceutical composition may comprise two populations of nanoparticles such as micelles, one comprising a first non-ionic surfactant and the resveratrol compound and the other population comprising a second non-ionic surfactant and the curcumin compound. The first and second non-ionic surfactants may be identical. Alternatively, the first and second non-ionic surfactant may be different.

In some embodiments, the nanoparticles may comprise the non-ionic surfactant and the resveratrol and/or curcumin compound at a suitable weight ratio, which lead to suitable particle sizes and/or suitable polydispersity index (PDI) values. For example, a suitable weight ratio between the resveratrol and/or curcumin compound and the non-ionic surfactant may range from about 1:1-1:1000, for example, between about 1:1-1:800, between about 1:1-1:600, between about 1:1-1:500, between about 1:1-1:200, or between about 1:1-1:100. In other examples, the suitable weight ratio between the resveratrol and/or curcumin compound and the non-ionic surfactant ranges from 1:5 to 1:1000, for example, between 1:5-1:150, between 1:5-1:200, between 1:5-1:300, between 1:5-1:400, between 1:5-1:500, between about 1:8-1:10, between about 1:8-1:20, between about 1:8-1:40, between about 1:8-1:100, between 1:8-1:150, between 1:8-1:200, between 1:8-1:300, between 1:8-1:400, between 1:8-1:500, between about 1:10-1:20, between about 1:10-1:40, between about 1:10-1:100, between 1:10-1:150, between 1:10-1:200, between 1:10-1:300, between 1:10-1:400, between 1:10-1:500, between about 1:20-1:40, between about 1:20-1:100, between 1:20-1:150, between 1:20-1:200, between 1:20-1:300, between 1:20-1:400, between 1:20-1:500, between about 1:40-1:100, between 1:40-1:150, between 1:40-1:200, between 1:40-1:300, between 1:40-1:400, between 1:40-1:500, between 1:100-1:150, between 1:100-1:200, between 1:100-1:300, between 1:100-1:400, between 1:100-1:500, between 1:150-1:200, between 1:150-1:300, between 1:150-1:400, or between 1:150-1:500.

The term “about” or “approximately” means within an acceptable error range for the particular value as determined by one of ordinary skill in the art, which will depend in part on how the value is measured or determined, i.e., the limitations of the measurement system. For example, “about” can mean within an acceptable standard deviation, per the practice in the art. Alternatively, “about” can mean a range of up to +20%, preferably up to +10%, more preferably up to +5%, and more preferably still up to 1% of a given value. Alternatively, particularly with respect to biological systems or processes, the term can mean within an order of magnitude, preferably within 2-fold, of a value. Where particular values are described in the application and claims, unless otherwise stated, the term “about” is implicit and in this context means within an acceptable error range for the particular value. In some embodiments, the hinge domain is a hinge domain of a naturally occurring protein.

In some instances, the pharmaceutical compositions disclosed herein may comprise nanoparticles having an average size of up to 200 nm, for example, up to 150 nm, up to 100 nm, up to 50 nm, or up to 20 nm. In some examples, the average size of the nanoparticles may range from 5-200 nm, for example, 5-150 nm, 5-100 nm, 5-50 nm, or 5-20 nm.

Alternatively or in addition, the nanoparticles in the pharmaceutical compositions may have a suitable PDI value, for example, up to 0.4. In some examples, the PDI value may range from 0.05-0.4, e.g., 0.05-0.35, 0.05-0.3, 0.05-0.25, 0.05-0.2, 0.05-0.15, or 0.05-0.1.

Any of the nanoparticles disclosed herein can be prepared by conventional methods or as disclosed herein. One example is provided below. A suitable amount of an active agent or a hydrophilic therapeutic agent may be mixed with a suitable solvent (e.g., alcohols (e.g., methanol, ethanol, propanol), acetonitrile, chlorinated solvents (e.g., dichloromethane, chloroform) diethyl ether, and ethyl acetate) and the mixture can be stirred (e.g., at 150-500 rpm) at a suitable temperature until the active agent or the hydrophilic therapeutic agent as disclosed herein is full dissolved in the solvent to form a solution. A suitable amount of a pharmaceutically acceptable non-ionic surfactant (e.g., those disclosed herein) can be added to the solution. The resultant mixture can be stirred (e.g., at 100-300 rpm) under a suitable temperature to volatilize the solvent. Once the solvent is completely volatilized, a suitable amount of a pharmaceutically acceptable aqueous solution (e.g., normal saline) can be added to the mixture to produce micelles having the active agent or hydrophilic agent encapsulated. The resultant particles can be filtered through a suitable filter (e.g., a 0.2 μm filter) and the filtered solution comprising drug-containing micelles can be stored in the dark in a refrigerator for future use.

Additional details on the preparation of the micelles of the present technology can be found in U.S. Pat. No. 10,610,496, the relevant disclosures of which are incorporated by reference herein for the subject matter and purpose referenced herein.

### (vi) Additional Components of Pharmaceutical Compositions

In some embodiments, the pharmaceutical composition disclosed herein may further comprise a co-solvent (e.g., to increase the solubility of drugs), a suspending agent (e.g., to reduce the sedimentation rate of drugs), an oil phase excipient (e.g., to increase the stability of the pharmaceutical composition and the solubility of drugs), an antimicrobial preservative, or a combination thereof.

In some instances, the co-solvent can comprise polyethylene glycol, propylene glycol, ethanol, and other co-solvents, or a combination thereof. Alternatively or in addition, the suspending agent may comprise sodium alginate, glycerol, carboxymethyl cellulose sodium, mannitol, and other suspending agents, or a combination thereof.

In some instances, the oil phase excipient may comprise unsaturated fatty acids, glycerol, triglycerides, and other oil phase excipients, or a combination thereof. For example, the oil phase excipient may comprise one or more unsaturated fatty acids, which may be oleic acid, castor oil, sesame oil, cottonseed oil, soybean oil, safflower oil, corn oil, and other unsaturated fatty acids, or a combination thereof. In some examples, the oil phase excipient may comprise triglycerides, which may be medium chain triglycerides.

In some instances, the pharmaceutical composition disclosed herein may be free of unsaturated fatty acids. Alternatively, or in addition, the pharmaceutical composition disclosed herein may be free of saturated fatty acids. In other examples, the pharmaceutical composition disclosed herein may be free of glycerol and/or triglycerides. In some examples, the pharmaceutical composition disclosed herein contains the non-ionic surfactant disclosed herein and is free of any additional oil phase excipients.

In some embodiments, the pharmaceutical composition disclosed herein may comprise a local anesthetic. Examples include, but are not limited to, amides, para-aminobenzoic acid esters, and amino ethers, or a combination thereof. In some examples, the local anesthetic comprises amides, which may bedibucaine, lidocaine, mepivacaine HCl, bupivacine HCl, pyrrocaine HCl, Prilocaine HCl, digammacaine, and oxethazaine, or a combination thereof. In other examples, the local anesthetic comprises para-aminobenzoic acid esters, which may be butacaine, dimethocaine, and tutocaine, or a combination thereof. In yet other examples, the local anesthetic comprises amino ethers, which may be quinisocaine and pramocaine, or a combination thereof.

Alternatively or in addition, the pharmaceutical composition disclosed herein may comprise one or more antioxidant. Examples include, but are not limited to, beta-carotene, lutein, lycopene, bilirubin, vitamin A, vitamin C (ascorbic acid), vitamin E, citric acid, sodium thiosulfate, Propyl gallate, uric acid, nitric oxide, nitroxide, pyruvate, catalase, superoxide dismutase, glutathione peroxidases, N-acetyl cysteine, and naringenin, or a combination thereof. Alternatively, the pharmaceutical composition is free of antioxidants.

Alternatively or in addition, the pharmaceutical compositions provided herein may further comprise a second active agent, which is not a resveratrol and/or curcumin compound disclosed herein. In some instances, the second active agent may be encapsulated in nanoparticles.

In some embodiments, the second active agent may comprise a hydrophilic therapeutic agent, for example, green tea extract, epicatechin, epicatechin gallate, epigallocatechin, gallocatechin gallate, gallocatechin, catechin gallate, catechin, epigallocatechin gallate (EGCG), caffeine, carnitine, L-carnitine, synephrine, chlorogenic acid, or a combination thereof.

In some embodiments, the pharmaceutical compositions described herein may be formulated as a topical formulation, for example, a cream, lotion, or gel for topical application. Such cream, lotion, or gel may be formulated using ingredients known in the art to be appropriate for topical medications. In some examples, the topical formation may be in the form of a transdermal patch.

Alternatively, the pharmaceutical compositions may consist essential of the biologically active agent, the osmolality adjusting agent, the non-ionic surfactant, and optionally water. Examples of such pharmaceutical compositions are provided in Example 1 and Example 2 below.

(vii) Exemplary Pharmaceutical Compositions

In some embodiments, the pharmaceutical composition comprises a curcumin compound such as curcumin and its pH value may range from about 5 to about 8 and the osmolality value of the pharmaceutical composition is 250-350 mOsm/kg.

In some embodiments, the pharmaceutical composition comprises a curcumin compound such as curcumin and its pH value may range from about 5 to about 8 and the osmolality value of the pharmaceutical composition is 270-330 mOsm/kg.

In some embodiments, the pharmaceutical composition comprises a curcumin compound such as curcumin and its pH value may range from about 5 to about 8 and the osmolality value of the pharmaceutical composition is 280-320 mOsm/kg.

In some embodiments, the pharmaceutical composition comprises a curcumin compound such as curcumin and its pH value may range from about 5 to about 7.5 and the osmolality value of the pharmaceutical composition is 250-350 mOsm/kg.

In some embodiments, the pharmaceutical composition comprises a curcumin compound such as curcumin and its pH value may range from about 5 to about 7.5 and the osmolality value of the pharmaceutical composition is 270-330 mOsm/kg.

In some embodiments, the pharmaceutical composition comprises a curcumin compound such as curcumin and its pH value may range from about 5 to about 7.5 and the osmolality value of the pharmaceutical composition is 280-320 mOsm/kg.

In some embodiments, the pharmaceutical composition comprises a curcumin compound such as curcumin and its pH value may range from about 5 to about 7 and the osmolality value of the pharmaceutical composition is 250-350 mOsm/kg.

In some embodiments, the pharmaceutical composition comprises a curcumin compound such as curcumin and its pH value may range from about 5 to about 7 and the osmolality value of the pharmaceutical composition is 270-330 mOsm/kg.

In some embodiments, the pharmaceutical composition comprises a curcumin compound such as curcumin and its pH value may range from about 5 to about 7 and the osmolality value of the pharmaceutical composition is 280-320 mOsm/kg.

In some embodiments, the pharmaceutical composition comprises a curcumin compound such as curcumin and its pH value may range from about 5 to about 6.5 and the osmolality value of the pharmaceutical composition is 250-350 mOsm/kg.

In some embodiments, the pharmaceutical composition comprises a curcumin compound such as curcumin and its pH value may range from about 5 to about 6.5 and the osmolality value of the pharmaceutical composition is 270-330 mOsm/kg.

In some embodiments, the pharmaceutical composition comprises a curcumin compound such as curcumin and its pH value may range from about 5 to about 6.5 and the osmolality value of the pharmaceutical composition is 280-320 mOsm/kg.

In some embodiments, the pharmaceutical composition comprises a curcumin compound such as curcumin and its pH value may range from about 5 to about 6 and the osmolality value of the pharmaceutical composition is 250-350 mOsm/kg.

In some embodiments, the pharmaceutical composition comprises a curcumin compound such as curcumin and its pH value may range from about 5 to about 6 and the osmolality value of the pharmaceutical composition is 270-330 mOsm/kg.

In some embodiments, the pharmaceutical composition comprises a curcumin compound such as curcumin and its pH value may range from about 5 to about 6 and the osmolality value of the pharmaceutical composition is 280-320 mOsm/kg.

In some embodiments, the pharmaceutical composition comprises a curcumin compound such as curcumin and its pH value may range from about 5.5 to about 8 and the osmolality value of the pharmaceutical composition is 250-350 mOsm/kg.

In some embodiments, the pharmaceutical composition comprises a curcumin compound such as curcumin and its pH value may range from about 5.5 to about 8 and the osmolality value of the pharmaceutical composition is 270-330 mOsm/kg.

In some embodiments, the pharmaceutical composition comprises a curcumin compound such as curcumin and its pH value may range from about 5.5 to about 8 and the osmolality value of the pharmaceutical composition is 280-320 mOsm/kg.

In some embodiments, the pharmaceutical composition comprises a curcumin compound such as curcumin and its pH value may range from about 6 to about 8 and the osmolality value of the pharmaceutical composition is 250-350 mOsm/kg.

In some embodiments, the pharmaceutical composition comprises a curcumin compound such as curcumin and its pH value may range from about 6 to about 8 and the osmolality value of the pharmaceutical composition is 270-330 mOsm/kg.

In some embodiments, the pharmaceutical composition comprises a curcumin compound such as curcumin and its pH value may range from about 6 to about 8 and the osmolality value of the pharmaceutical composition is 280-320 mOsm/kg.

In some embodiments, the pharmaceutical composition comprises a curcumin compound such as curcumin and its pH value may range from about 6.5 to about 8 and the osmolality value of the pharmaceutical composition is 250-350 mOsm/kg.

In some embodiments, the pharmaceutical composition comprises a curcumin compound such as curcumin and its pH value may range from about 6.5 to about 8 and the osmolality value of the pharmaceutical composition is 270-330 mOsm/kg.

In some embodiments, the pharmaceutical composition comprises a curcumin compound such as curcumin and its pH value may range from about 6.5 to about 8 and the osmolality value of the pharmaceutical composition is 280-320 mOsm/kg.

In some embodiments, the pharmaceutical composition comprises a curcumin compound such as curcumin and its pH value may range from about 7 to about 8 and the osmolality value of the pharmaceutical composition is 250-350 mOsm/kg.

In some embodiments, the pharmaceutical composition comprises a curcumin compound such as curcumin and its pH value may range from about 7 to about 8 and the osmolality value of the pharmaceutical composition is 270-330 mOsm/kg.

In some embodiments, the pharmaceutical composition comprises a curcumin compound such as curcumin and its pH value may range from about 7 to about 8 and the osmolality value of the pharmaceutical composition is 280-320 mOsm/kg.

In some embodiments, the pharmaceutical composition comprises a curcumin compound such as curcumin and its pH value may range from about 5.5 to about 7.5 and the osmolality value of the pharmaceutical composition is 250-350 mOsm/kg.

In some embodiments, the pharmaceutical composition comprises a curcumin compound such as curcumin and its pH value may range from about 5.5 to about 7.5 and the osmolality value of the pharmaceutical composition is 270-330 mOsm/kg.

In some embodiments, the pharmaceutical composition comprises a curcumin compound such as curcumin and its pH value may range from about 5.5 to about 7.5 and the osmolality value of the pharmaceutical composition is 280-320 mOsm/kg.

In some embodiments, the pharmaceutical composition comprises a curcumin compound such as curcumin and its pH value may range from about 6.5 to about 7.5 and the osmolality value of the pharmaceutical composition is 250-350 mOsm/kg.

In some embodiments, the pharmaceutical composition comprises a curcumin compound such as curcumin and its pH value may range from about 6.5 to about 7.5 and the osmolality value of the pharmaceutical composition is 270-330 mOsm/kg.

In some embodiments, the pharmaceutical composition comprises a curcumin compound such as curcumin and its pH value may range from about 6.5 to about 7.5 and the osmolality value of the pharmaceutical composition is 280-320 mOsm/kg.

In some embodiments, the pharmaceutical composition comprises a curcumin compound such as curcumin and its pH value may range from about 5.5 to about 6.5 and the osmolality value of the pharmaceutical composition is 250-350 mOsm/kg.

In some embodiments, the pharmaceutical composition comprises a curcumin compound such as curcumin and its pH value may range from about 5.5 to about 6.5 and the osmolality value of the pharmaceutical composition is 270-330 mOsm/kg.

In some embodiments, the pharmaceutical composition comprises a curcumin compound such as curcumin and its pH value may range from about 5.5 to about 6.5 and the osmolality value of the pharmaceutical composition is 280-320 mOsm/kg.

In some embodiments, the pharmaceutical composition comprises a resveratrol compound such as resveratrol and its pH value may range from about 4.5 to about 8 and the osmolality value of the pharmaceutical composition is 250-350 mOsm/kg.

In some embodiments, the pharmaceutical composition comprises a resveratrol compound such as resveratrol and its pH value may range from about 4.5 to about 8 and the osmolality value of the pharmaceutical composition is 270-330 mOsm/kg.

In some embodiments, the pharmaceutical composition comprises a resveratrol compound such as resveratrol and its pH value may range from about 4.5 to about 8 and the osmolality value of the pharmaceutical composition is 280-320 mOsm/kg.

In some embodiments, the pharmaceutical composition comprises a resveratrol compound such as resveratrol and its pH value may range from about 5 to about 8 and the osmolality value of the pharmaceutical composition is 250-350 mOsm/kg.

In some embodiments, the pharmaceutical composition comprises a resveratrol compound such as resveratrol and its pH value may range from about 5 to about 8 and the osmolality value of the pharmaceutical composition is 270-330 mOsm/kg.

In some embodiments, the pharmaceutical composition comprises a resveratrol compound such as resveratrol and its pH value may range from about 5 to about 8 and the osmolality value of the pharmaceutical composition is 280-320 mOsm/kg.

In some embodiments, the pharmaceutical composition comprises a resveratrol compound such as resveratrol and its pH value may range from about 5.5 to about 8 and the osmolality value of the pharmaceutical composition is 250-350 mOsm/kg.

In some embodiments, the pharmaceutical composition comprises a resveratrol compound such as resveratrol and its pH value may range from about 5.5 to about 8 and the osmolality value of the pharmaceutical composition is 270-330 mOsm/kg.

In some embodiments, the pharmaceutical composition comprises a resveratrol compound such as resveratrol and its pH value may range from about 5.5 to about 8 and the osmolality value of the pharmaceutical composition is 280-320 mOsm/kg.

In some embodiments, the pharmaceutical composition comprises a resveratrol compound such as resveratrol and its pH value may range from about 6 to about 8 and the osmolality value of the pharmaceutical composition is 250-350 mOsm/kg.

In some embodiments, the pharmaceutical composition comprises a resveratrol compound such as resveratrol and its pH value may range from about 6 to about 8 and the osmolality value of the pharmaceutical composition is 270-330 mOsm/kg.

In some embodiments, the pharmaceutical composition comprises a resveratrol compound such as resveratrol and its pH value may range from about 6 to about 8 and the osmolality value of the pharmaceutical composition is 280-320 mOsm/kg.

In some embodiments, the pharmaceutical composition comprises a resveratrol compound such as resveratrol and its pH value may range from about 6.5 to about 8 and the osmolality value of the pharmaceutical composition is 250-350 mOsm/kg.

In some embodiments, the pharmaceutical composition comprises a resveratrol compound such as resveratrol and its pH value may range from about 6.5 to about 8 and the osmolality value of the pharmaceutical composition is 270-330 mOsm/kg.

In some embodiments, the pharmaceutical composition comprises a resveratrol compound such as resveratrol and its pH value may range from about 6.5 to about 8 and the osmolality value of the pharmaceutical composition is 280-320 mOsm/kg.

In some embodiments, the pharmaceutical composition comprises a resveratrol compound such as resveratrol and its pH value may range from about 7 to about 8 and the osmolality value of the pharmaceutical composition is 250-350 mOsm/kg.

In some embodiments, the pharmaceutical composition comprises a resveratrol compound such as resveratrol and its pH value may range from about 7 to about 8 and the osmolality value of the pharmaceutical composition is 270-330 mOsm/kg.

In some embodiments, the pharmaceutical composition comprises a resveratrol compound such as resveratrol and its pH value may range from about 7 to about 8 and the osmolality value of the pharmaceutical composition is 280-320 mOsm/kg.

In some embodiments, the pharmaceutical composition comprises a resveratrol compound such as resveratrol and its pH value may range from about 6.5 to about 7.5 and the osmolality value of the pharmaceutical composition is 250-350 mOsm/kg.

In some embodiments, the pharmaceutical composition comprises a resveratrol compound such as resveratrol and its pH value may range from about 6.5 to about 7.5 and the osmolality value of the pharmaceutical composition is 270-330 mOsm/kg.

In some embodiments, the pharmaceutical composition comprises a resveratrol compound such as resveratrol and its pH value may range from about 6.5 to about 7.5 and the osmolality value of the pharmaceutical composition is 280-320 mOsm/kg.

In some embodiments, the pharmaceutical composition comprises a resveratrol compound such as resveratrol and its pH value may range from about 5 to about 7.5 and the osmolality value of the pharmaceutical composition is 250-350 mOsm/kg.

In some embodiments, the pharmaceutical composition comprises a resveratrol compound such as resveratrol and its pH value may range from about 5 to about 7.5 and the osmolality value of the pharmaceutical composition is 270-330 mOsm/kg.

In some embodiments, the pharmaceutical composition comprises a resveratrol compound such as resveratrol and its pH value may range from about 5 to about 7.5 and the osmolality value of the pharmaceutical composition is 280-320 mOsm/kg.

In some embodiments, the pharmaceutical composition comprises a resveratrol compound such as resveratrol and its pH value may range from about 5.5 to about 7 and the osmolality value of the pharmaceutical composition is 250-350 mOsm/kg.

In some embodiments, the pharmaceutical composition comprises a resveratrol compound such as resveratrol and its pH value may range from about 5.5 to about 7 and the osmolality value of the pharmaceutical composition is 270-330 mOsm/kg.

In some embodiments, the pharmaceutical composition comprises a resveratrol compound such as resveratrol and its pH value may range from about 5.5 to about 7 and the osmolality value of the pharmaceutical composition is 280-320 mOsm/kg.

In some embodiments, the pharmaceutical composition comprises a curcumin compound such as curcumin and a resveratrol compound such as resveratrol and its pH value may range from about 5 to about 8.5 and the osmolality value of the pharmaceutical composition is 250-350 mOsm/kg.

In some embodiments, the pharmaceutical composition comprises a curcumin compound such as curcumin and a resveratrol compound such as resveratrol and its pH value may range from about 5 to about 8.5 and the osmolality value of the pharmaceutical composition is 270-330 mOsm/kg.

In some embodiments, the pharmaceutical composition comprises a curcumin compound such as curcumin and a resveratrol compound such as resveratrol and its pH value may range from about 5 to about 8.5 and the osmolality value of the pharmaceutical composition is 280-320 mOsm/kg.

In some embodiments, the pharmaceutical composition comprises a curcumin compound such as curcumin and a resveratrol compound such as resveratrol and its pH value may range from about 5 to about 8 and the osmolality value of the pharmaceutical composition is 250-350 mOsm/kg.

In some embodiments, the pharmaceutical composition comprises a curcumin compound such as curcumin and a resveratrol compound such as resveratrol and its pH value may range from about 5 to about 8 and the osmolality value of the pharmaceutical composition is 270-330 mOsm/kg.

In some embodiments, the pharmaceutical composition comprises a curcumin compound such as curcumin and a resveratrol compound such as resveratrol and its pH value may range from about 5 to about 8 and the osmolality value of the pharmaceutical composition is 280-320 mOsm/kg.

In some embodiments, the pharmaceutical composition comprises a curcumin compound such as curcumin and a resveratrol compound such as resveratrol and its pH value may range from about 5 to about 7.5 and the osmolality value of the pharmaceutical composition is 250-350 mOsm/kg.

In some embodiments, the pharmaceutical composition comprises a curcumin compound such as curcumin and a resveratrol compound such as resveratrol and its pH value may range from about 5 to about 7.5 and the osmolality value of the pharmaceutical composition is 270-330 mOsm/kg.

In some embodiments, the pharmaceutical composition comprises a curcumin compound such as curcumin and a resveratrol compound such as resveratrol and its pH value may range from about 5 to about 7.5 and the osmolality value of the pharmaceutical composition is 280-320 mOsm/kg.

In some embodiments, the pharmaceutical composition comprises a curcumin compound such as curcumin and a resveratrol compound such as resveratrol and its pH value may range from about 5 to about 7 and the osmolality value of the pharmaceutical composition is 250-350 mOsm/kg.

In some embodiments, the pharmaceutical composition comprises a curcumin compound such as curcumin and a resveratrol compound such as resveratrol and its pH value may range from about 5 to about 7 and the osmolality value of the pharmaceutical composition is 270-330 mOsm/kg.

In some embodiments, the pharmaceutical composition comprises a curcumin compound such as curcumin and a resveratrol compound such as resveratrol and its pH value may range from about 5 to about 7 and the osmolality value of the pharmaceutical composition is 280-320 mOsm/kg.

In some embodiments, the pharmaceutical composition comprises a curcumin compound such as curcumin and a resveratrol compound such as resveratrol and its pH value may range from about 5 to about 6.5 and the osmolality value of the pharmaceutical composition is 250-350 mOsm/kg.

In some embodiments, the pharmaceutical composition comprises a curcumin compound such as curcumin and a resveratrol compound such as resveratrol and its pH value may range from about 5 to about 6.5 and the osmolality value of the pharmaceutical composition is 270-330 mOsm/kg.

In some embodiments, the pharmaceutical composition comprises a curcumin compound such as curcumin and a resveratrol compound such as resveratrol and its pH value may range from about 5 to about 6.5 and the osmolality value of the pharmaceutical composition is 280-320 mOsm/kg.

In some embodiments, the pharmaceutical composition comprises a curcumin compound such as curcumin and a resveratrol compound such as resveratrol and its pH value may range from about 5 to about 6 and the osmolality value of the pharmaceutical composition is 250-350 mOsm/kg.

In some embodiments, the pharmaceutical composition comprises a curcumin compound such as curcumin and a resveratrol compound such as resveratrol and its pH value may range from about 5 to about 6 and the osmolality value of the pharmaceutical composition is 270-330 mOsm/kg.

In some embodiments, the pharmaceutical composition comprises a curcumin compound such as curcumin and a resveratrol compound such as resveratrol and its pH value may range from about 5 to about 6 and the osmolality value of the pharmaceutical composition is 280-320 mOsm/kg.

In some embodiments, the pharmaceutical composition comprises a curcumin compound such as curcumin and a resveratrol compound such as resveratrol and its pH value may range from about 5.5 to about 8.5 and the osmolality value of the pharmaceutical composition is 250-350 mOsm/kg.

In some embodiments, the pharmaceutical composition comprises a curcumin compound such as curcumin and a resveratrol compound such as resveratrol and its pH value may range from about 5.5 to about 8.5 and the osmolality value of the pharmaceutical composition is 270-330 mOsm/kg.

In some embodiments, the pharmaceutical composition comprises a curcumin compound such as curcumin and a resveratrol compound such as resveratrol and its pH value may range from about 5.5 to about 8.5 and the osmolality value of the pharmaceutical composition is 280-320 mOsm/kg.

In some embodiments, the pharmaceutical composition comprises a curcumin compound such as curcumin and a resveratrol compound such as resveratrol and its pH value may range from about 5.5 to about 8 and the osmolality value of the pharmaceutical composition is 250-350 mOsm/kg.

In some embodiments, the pharmaceutical composition comprises a curcumin compound such as curcumin and a resveratrol compound such as resveratrol and its pH value may range from about 5.5 to about 8 and the osmolality value of the pharmaceutical composition is 270-330 mOsm/kg.

In some embodiments, the pharmaceutical composition comprises a curcumin compound such as curcumin and a resveratrol compound such as resveratrol and its pH value may range from about 5.5 to about 8 and the osmolality value of the pharmaceutical composition is 280-320 mOsm/kg.

In some embodiments, the pharmaceutical composition comprises a curcumin compound such as curcumin and a resveratrol compound such as resveratrol and its pH value may range from about 6 to about 8 and the osmolality value of the pharmaceutical composition is 250-350 mOsm/kg.

In some embodiments, the pharmaceutical composition comprises a curcumin compound such as curcumin and a resveratrol compound such as resveratrol and its pH value may range from about 6 to about 8 and the osmolality value of the pharmaceutical composition is 270-330 mOsm/kg.

In some embodiments, the pharmaceutical composition comprises a curcumin compound such as curcumin and a resveratrol compound such as resveratrol and its pH value may range from about 6 to about 8 and the osmolality value of the pharmaceutical composition is 280-320 mOsm/kg.

In some embodiments, the pharmaceutical composition comprises a curcumin compound such as curcumin and a resveratrol compound such as resveratrol and its pH value may range from about 6.5 to about 8 and the osmolality value of the pharmaceutical composition is 250-350 mOsm/kg.

In some embodiments, the pharmaceutical composition comprises a curcumin compound such as curcumin and a resveratrol compound such as resveratrol and its pH value may range from about 6.5 to about 8 and the osmolality value of the pharmaceutical composition is 270-330 mOsm/kg.

In some embodiments, the pharmaceutical composition comprises a curcumin compound such as curcumin and a resveratrol compound such as resveratrol and its pH value may range from about 6.5 to about 8 and the osmolality value of the pharmaceutical composition is 280-320 mOsm/kg.

In some embodiments, the pharmaceutical composition comprises a curcumin compound such as curcumin and a resveratrol compound such as resveratrol and its pH value may range from about 7 to about 8 and the osmolality value of the pharmaceutical composition is 250-350 mOsm/kg.

In some embodiments, the pharmaceutical composition comprises a curcumin compound such as curcumin and a resveratrol compound such as resveratrol and its pH value may range from about 7 to about 8 and the osmolality value of the pharmaceutical composition is 270-330 mOsm/kg.

In some embodiments, the pharmaceutical composition comprises a curcumin compound such as curcumin and a resveratrol compound such as resveratrol and its pH value may range from about 7 to about 8 and the osmolality value of the pharmaceutical composition is 280-320 mOsm/kg.

In some embodiments, the pharmaceutical composition comprises a curcumin compound such as curcumin and a resveratrol compound such as resveratrol and its pH value may range from about 5.5 to about 7 and the osmolality value of the pharmaceutical composition is 250-350 mOsm/kg.

In some embodiments, the pharmaceutical composition comprises a curcumin compound such as curcumin and a resveratrol compound such as resveratrol and its pH value may range from about 5.5 to about 7 and the osmolality value of the pharmaceutical composition is 270-330 mOsm/kg.

In some embodiments, the pharmaceutical composition comprises a curcumin compound such as curcumin and a resveratrol compound such as resveratrol and its pH value may range from about 5.5 to about 7 and the osmolality value of the pharmaceutical composition is 280-320 mOsm/kg.

In some embodiments, the pharmaceutical composition comprises a curcumin compound such as curcumin and a resveratrol compound such as resveratrol and its pH value may range from about 5.5 to about 6.5 and the osmolality value of the pharmaceutical composition is 250-350 mOsm/kg.

In some embodiments, the pharmaceutical composition comprises a curcumin compound such as curcumin and a resveratrol compound such as resveratrol and its pH value may range from about 5.5 to about 6.5 and the osmolality value of the pharmaceutical composition is 270-330 mOsm/kg.

In some embodiments, the pharmaceutical composition comprises a curcumin compound such as curcumin and a resveratrol compound such as resveratrol and its pH value may range from about 5.5 to about 6.5 and the osmolality value of the pharmaceutical composition is 280-320 mOsm/kg.

### II. Methods for Therapeutic Applications

Any of the pharmaceutical compositions disclosed herein, comprising the resveratrol compound such as resveratrol, the curcumin compound such as curcumin, or both, can be used to for therapeutic applications, for example, to lower fat volume in a treated area or body, to alleviate a condition caused by abnormal subcutaneous deposit of adipose tissue or fat.

As used herein, “an effective amount” refers to the amount of each active agent required to confer therapeutic effect on the subject, either alone or in combination with one or more other active agents. Determination of whether an amount of the pharmaceutical composition achieved the therapeutic effect would be evident to one of skill in the art. Effective amounts vary, as recognized by those skilled in the art, depending on the particular condition being treated, the severity of the condition, the individual patient parameters including age, physical condition, size, gender and weight, the duration of the treatment, the nature of concurrent therapy (if any), the specific route of administration and like factors within the knowledge and expertise of the health practitioner. These factors are well known to those of ordinary skill in the art and can be addressed with no more than routine experimentation. It is generally preferred that a maximum dose of the individual components or combinations thereof be used, that is, the highest safe dose according to sound medical judgment.

Empirical considerations, such as the half-life, generally will contribute to the determination of the dosage. Frequency of administration may be determined and adjusted over the course of therapy, and is generally, but not necessarily, based on treatment and/or suppression and/or amelioration and/or delay of a target disease/disorder. Alternatively, sustained continuous release formulations may be appropriate. Various formulations and devices for achieving sustained release are known in the art.

For the purpose of the present disclosure, the appropriate dosage of the pharmaceutical composition as described herein will depend on the specific active agents and optionally hydrophilic agents employed, the type and severity of the disease/disorder, whether the composition is administered for preventive or therapeutic purposes, previous therapy, the patient's clinical history and response to the active agents, and the discretion of the attending physician. Typically, the clinician will administer a pharmaceutical composition disclosed herein, until a dosage is reached that achieves the desired result.

In some embodiments, the desired result is the reduction of fat volume in injected area. Methods of determining whether a dosage resulted in the desired result would be evident to one of skill in the art. The particular dosage regimen such as dose, timing and repetition, used in the method described herein will depend on the particular subject and that subject's medical history. Treatment efficacy for the target disease/disorder can be assessed by methods well-known in the art.

As used herein, the term “treating” refers to the application or administration of a composition including one or more active agents to a subject, who has a target disease or disorder, a symptom of the disease/disorder, or a predisposition toward the disease/disorder, with the purpose to cure, heal, alleviate, relieve, alter, remedy, ameliorate, improve, or affect the disorder, the symptom of the disease, or the predisposition toward the disease or disorder.

Alleviating a target disease/disorder includes delaying the development or progression of the disease or reducing disease severity or prolonging survival. Alleviating the disease or prolonging survival does not necessarily require curative results. As used therein, “delaying” the development of a target disease or disorder means to defer, hinder, slow, retard, stabilize, and/or postpone progression of the disease. This delay can be of varying lengths of time, depending on the history of the disease and/or individuals being treated. A method that delays or alleviates the development of a disease, or delays the onset of the disease, is a method that reduces probability of developing one or more symptoms of the disease in a given time frame and/or reduces extent of the symptoms in a given time frame, when compared to not using the method. Such comparisons are typically based on clinical studies, using a number of subjects sufficient to give a statistically significant result.

“Development” or “progression” of a disease means initial manifestations and/or ensuing progression of the disease. Development of the disease can be detectable and assessed using standard clinical techniques as well known in the art. However, development also refers to progression that may be undetectable. For purpose of this disclosure, development or progression refers to the biological course of the symptoms. “Development” includes occurrence, recurrence, and onset. As used herein “onset” or “occurrence” of a target disease or disorder includes initial onset and/or recurrence.

Any of the pharmaceutical compositions disclosed herein may be administered to the subject 1-4 times every day for a suitable period of time, for example, 1 month to 1 year. Duration of the treatment may depend on the treatment results.

In some embodiments, the pharmaceutical composition disclosed herein is for use to reduce fat and/or adipocytes (e.g., reduce local fat), thereby benefiting treatment of diseases associated with such (e.g., abnormal deposit of fat in certain tissues). To perform the treatment method provided herein, an effective amount of the pharmaceutical composition may be administered to a subject in need of the treatment via a suitable route such as those provided herein. In some instances, the subject may be a human patient having or suspected of having a target disease or condition. Exemplary target diseases and disorders include Dercum's disease (Adiposis dolorosa), Gardner syndrome, Familial multiple lipomatosis (Hereditary multiple lipomatosis), Madelung's disease (multiple symmetric lipomatosis), painful lipomas, angiolipomas, lipomatosis, fat disorder, subcutaneous adipose tissue diseases, lipedema, skin disease, a lipid metabolism disorder, a metabolic disease, rare adipose disorders (RADs), Panniculitis, Weber-Christian disease, Progressive lipodystrophy or other diseases/symptoms related to fatty lesions. In some instances, the composition provided herein can be used to reduce local fat (e.g., subcutaneous fat, visceral fat, or fat in respiratory tract) and/or reduce body weight.

### III. Kits for Use for Therapeutic Application

The present disclosure also provides kits for use in the contemplated therapeutic uses. Such kits can include one or more containers comprising the pharmaceutical composition disclosed herein. In some instances, the pharmaceutical composition may be co-used with a second therapeutic agent.

In some embodiments, the kit can comprise instructions for use in accordance with any of the methods described herein. The included instructions can comprise a description of administration of the pharmaceutical composition, and optionally the second therapeutic agent, to treat. The instructions relating to the use of the pharmaceutical composition disclosed herein generally include information as to dosage, dosing schedule, and route of administration for the intended treatment. The containers may be unit doses, bulk packages (e.g., multi-dose packages) or sub-unit doses. Instructions supplied in the kits of the invention are typically written instructions on a label or package insert (e.g., a paper sheet included in the kit), but machine-readable instructions (e.g., instructions carried on a magnetic or optical storage disk) are also acceptable.

The label or package insert indicates that the composition is used for treating local fat volumes. Instructions may be provided for practicing any of the methods described herein.

The kits of this invention are in suitable packaging. Suitable packaging includes, but is not limited to, vials, bottles, jars, flexible packaging (e.g., sealed Mylar or plastic bags), and the like. Also contemplated are packages for use in combination with a specific device, such as an inhaler, nasal administration device (e.g., an atomizer) or an infusion device such as a minipump. A kit may have a sterile access port (for example the container may be an intravenous solution bag or a vial having a stopper pierceable by a hypodermic injection needle). The container may also have a sterile access port (for example the container may be an intravenous solution bag or a vial having a stopper pierceable by a hypodermic injection needle).

Kits may optionally provide additional components such as buffers and interpretive information. Normally, the kit comprises a container and a label or package insert(s) on or associated with the container. In some embodiments, the invention provides articles of manufacture comprising contents of the kits described above.

### General Techniques

The practice of the present disclosure will employ, unless otherwise indicated, conventional techniques of molecular biology (including recombinant techniques), microbiology, cell biology, biochemistry, and immunology, which are within the skill of the art. Such techniques are explained fully in the literature, such as Molecular Cloning: A Laboratory Manual, second edition (Sambrook, et al., 1989) Cold Spring Harbor Press; Oligonucleotide Synthesis (M. J. Gait, ed. 1984); Methods in Molecular Biology, Humana Press; Cell Biology: A Laboratory Notebook (J. E. Cellis, ed., 1989) Academic Press; Animal Cell Culture (R. I. Freshney, ed. 1987); Introuction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds. 1993-8) J. Wiley and Sons; Methods in Enzymology (Academic Press, Inc.); Handbook of Experimental Immunology (D. M. Weir and C. C. Blackwell, eds.): Gene Transfer Vectors for Mammalian Cells (J. M. Miller and M. P. Calos, eds., 1987); Current Protocols in Molecular Biology (F. M. Ausubel, et al. eds. 1987); PCR: The Polymerase Chain Reaction, (Mullis, et al., eds. 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: a practice approach (D. Catty., ed., IRL Press, 1988-1989); Monoclonal antibodies: a practical approach (P. Shepherd and C. Dean, eds., Oxford University Press, 2000); Using antibodies: a laboratory manual (E. Harlow and D. Lane (Cold Spring Harbor Laboratory Press, 1999); The Antibodies (M. Zanetti and J. D. Capra, eds. Harwood Academic Publishers, 1995); DNA Cloning: A practical Approach, Volumes I and II (D. N. Glover ed. 1985); Nucleic Acid Hybridization (B. D. Hames & S. J. Higgins eds. (1985; Transcription and Translation (B. D. Hames & S. J. Higgins, eds. (1984»; Animal Cell Culture (R. I. Freshney, ed. (1986»; Immobilized Cells and Enzymes (IRL Press, (1986»; and B. Perbal, A practical Guide To Molecular Cloning (1984); F. M. Ausubel et al. (eds.).

Without further elaboration, it is believed that one skilled in the art can, based on the above description, utilize the present invention to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### EXAMPLES

While the present disclosure has been described with reference to the specific embodiments thereof, it should be understood by those skilled in the art that various changes may be made, and equivalents may be substituted without departing from the true spirit and scope of the disclosure. In addition, many modifications may be made to adapt a particular situation, material, composition of matter, process, process step or steps, to the objective, spirit, and scope of the present disclosure. All such modifications are intended to be within the scope of the disclosure.

### Example 1: Assessment of pH and Osmolality in Exemplary Pharmaceutical Formulations

This example explores osmolality and pH values of formulations comprising curcumin and/or resveratrol, non-ionic surfactant (e.g., ELP or HS15), and various osmolality adjusting agents (e.g., NaCl, propylene glycol, mannitol, and Na₂HPO₄). Osmolality (mOsm/kg) and pH values for each pharmaceutical formulation listed in Tables 1-7 below were measured following routine methods.

TABLE 1
Control Group

| Group | OP-A-DW | OP-A1-DW | OP-A2-DW |
|---|---|---|---|
| Curcumin | 0.4% (4 mg/mL) | 0.4% (4 mg/mL) | — |
| Resveratrol | 0.1% (1 mg/mL) | — | 0.1% (1 mg/mL) |
| ELP | 20% | 16% | 10% |
| Water | 79.5% | 83.6% | 89.9% |
| Total | 100% | 100% | 100% |
| pH value | 4.80 | 5.05 | 5.35 |
| Osmolality | 95 | 54 | 23 |
| (mOsm/kg) | | | |

As shown in Table 1, the exemplary formulations without the osmolality adjusting agent exhibited low osmolality.

The control formulations listed in Table 1 above were diluted with normal saline (0.9% NaCl), resulting in the formulations listed in Table 2 below. The osmolality values of such diluted formulations are listed in Table 2 as well.

TABLE 2
Formulations Diluted with 0.9% Sodium Chloride

| Group | OP-A-20-NS | OP-A-25-NS | OP-A1-NS | OP-A2-NS |
|---|---|---|---|---|
| Curcumin | 0.4% (4 mg/mL) | 0.4% (4 mg/mL) | 0.4% (4 mg/mL) | — |
| Resveratrol | 0.1% (1 mg/mL) | 0.1% (1 mg/mL) | — | 0.1% (1 mg/mL) |
| ELP | 20% | 25% | 16% | 10% |
| NaCl | 0.72% | 0.67% | 0.75% | 0.81% |
| Water | 78.78% | 73.83% | 82.85% | 89.09% |
| Total | 100% | 100% | 100% | 100% |
| Osmolality | 446 | 592 | 395 | 343 |
| (mOsm/kg) | | | | |

Tables 3-6 list formulations with an osmolality adjusting agent and the corresponding osmolality and pH values.

TABLE 3
Formulations Containing Sodium Chloride (NaCl)

| Group | OP-A-NACL | OP-A1-NACL | OP-A2-NACL |
|---|---|---|---|
| Curcumin | 0.4% (4 mg/mL) | 0.4% (4 mg/mL) | — |
| Resveratrol | 0.1% (1 mg/mL) | — | 0.1% (1 mg/mL) |
| ELP | 20% | 16% | 10% |
| NaCl | 0.43% | 0.6% | 0.73% |
| Water | 79.07% | 83% | 89.17% |
| Total | 100% | 100% | 100% |
| pH value | 5.6 | 5.6 | 5.6 |
| Osmolality | 303 | 318 | 303 |
| (mOsm/kg) | | | |

TABLE 4
Formulations Containing Propylene Glycol

| Group | OP-A-PG | OP-A1-PG | OP-A2-PG |
|---|---|---|---|
| Curcumin | 0.4% (4 mg/mL) | 0.4% (4 mg/mL) | — |
| Resveratrol | 0.1% (1 mg/mL) | — | 0.1% (1 mg/mL) |
| ELP | 20% | 16% | 10% |
| Propylene glycol | 1.1% | 1.4% | 1.7% |
| Water | 78.9% | 82.6% | 88.3% |
| Total | 100% | 100% | 100% |
| pH value | 4.33 | 4.09 | 3.97 |
| Osmolality | 310 | 302 | 284 |
| (mOsm/kg) | | | |

TABLE 5
Formulations Containing Mannitol

| Group | OP-A-MAN | OP-A1-MAN | OP-A2-MAN |
|---|---|---|---|
| Curcumin | 0.4% (4 mg/mL) | 0.4% (4 mg/mL) | — |
| Resveratrol | 0.1% (1 mg/mL) | — | 0.1% (1 mg/mL) |
| ELP | 20% | 16% | 10% |
| Mannitol | 2% | 2.8% | 3.7% |
| Water | 78% | 81.2% | 86.3% |
| Total | 100% | 100% | 100% |
| pH value | 4.88 | 5.65 | 5.41 |
| Osmolality | 302 | 292 | 294 |
| (mOsm/kg) | | | |

TABLE 6
Formulations Containing Na₂HPO₄

| Group | OP-A-DSP | OP-A1-DSP | OP-A2-DSP |
|---|---|---|---|
| Curcumin | 0.4% (4 mg/mL) | 0.4% (4 mg/mL) | — |
| Resveratrol | 0.1% (1 mg/mL) | — | 0.1% (1 mg/mL) |
| ELP | 20% | 16% | 10% |
| Na₂HPO₄ | 0.6% | 0.9% | 1.3% |
| Water | 79.4% | 83.1% | 88.7% |
| Total | 100% | 100% | 100% |
| pH value | 8.14 | 8.18 | 8.35 |
| Osmolality | 294 | 292 | 313 |
| (mOsm/kg) | | | |

Tables 7 and 8 below list formulations with different non-ionic surfactants, including HS-15 and CS20. The corresponding osmolality values are also listed.

TABLE 7
Formulations Containing HS-15 (Polyoxyethylene Group)

| Group | OP-A-HS15 | OP-A1-HS15 | OP-A2-HS15 |
|---|---|---|---|
| Curcumin | 0.4% (4 mg/mL) | 0.4% (4 mg/mL) | — |
| Resveratrol | 0.1% (1 mg/mL) | — | 0.1% (1 mg/mL) |
| HS-15 | 20% | 16% | 10% |
| NaCl | 0.1% | 0.3% | 0.5% |
| Water | 79.9% | 83.7% | 89.5% |
| Total | 100% | 100% | 100% |
| pH value | 7.12 | 7.16 | 6.74 |
| Osmolality | 319 | 305 | 276 |
| (mOsm/kg) | | | |

TABLE 8
Formulations Containing CS 20 (Polyoxyethylene Group)

| Group | OP-A-CS20 | OP-A1-CS20 | OP-A2-CS20 |
|---|---|---|---|
| Curcumin | 0.4% (4 mg/mL) | 0.4% (4 mg/mL) | — |
| Resveratrol | 0.1% (1 mg/mL) | — | 0.1% (1 mg/mL) |
| CS 20 | 20% | 16% | 10% |
| NaCl | 0.4% | 0.5% | 0.7% |
| Water | 79.1% | 83.5% | 89.2% |
| Total | 100% | 100% | 100% |
| pH value | 5.73 | 5.70 | 5.65 |
| Osmolality | 309 | 281 | 300 |
| (mOsm/kg) | | | |

### Example 2: Evaluation of Lipolysis Efficacy of Exemplary Formulations in Animal Models

Diet-Induced Obesity (DIO) rats are an animal model widely used to study the effects weight loss and other beneficial metabolic properties for therapeutic agent candidates. Gijsen and Kotzé-Hörstmann., Obesity Research & Clinical Practice, 2023, 17(6):449-457. This example explores the therapeutic effects of an exemplary composition provided herein for reduction of gluteal fat in a DIO SD rat model.

Sprague Dawley (SD) rats were fed 60% high-fat diet (HFD) to induce obesity for 4 weeks. On the 10^{th} day, the rats are randomized into four groups, each being treated as described in Table 9 below. Prior to injecting the indicated treatment formulation, gluteal fat at the injection site was scanned by micro-CT (p-CT). In the third week of HFD induction, rats were injected with 4 mL/kg of normal saline for the control group (HFD-C), with 4 mL/kg of OP-A1 (the OP-A1 group), with 4 mL/kg of OP-A2 (the OP-A2 group), and with 4 mL/kg of OP-A (the OP-A group) via subcutaneous injection.

All groups were injected once a day for 4 consecutive days. The study design is summarized in Table 9 below. Details of the tested formulations are provided in Table 3 above.

TABLE 9
Study Design

| | | Diet- | | | | Dose | |
|---|---|---|---|---|---|---|---|
| Study | Animal | Induced | Injection | | Dose Level | Volume | Dose |
| Group | No. | Period | Site | Test Article | (mg/kg) | (mL/kg) | Frequency |
| HFD-C | 13 | 4 weeks | Gluteal Fat | Normal Saline | — | 4 | Once a day, |
| OP-A1 | 13 | | | OP-A1 | Equivalent dose | 4 | total 4 doses |
| | | | | | to OP-A | | |
| OP-A2 | 13 | | | OP-A2 | Equivalent dose | 4 | |
| | | | | | to OP-A | | |
| OP-A | 13 | | | OP-A | 20 | 4 | |

A second micro-CT ranging from pubis to the coccyx mainly covering the tail fat mass was performed 14 days after the final injection. Micro-CT analysis images were all gray-scale black and white images, and the gray-scale threshold range was 0-255. The test conditions set were the lower gray threshold of adipose tissue to 40 and the upper gray threshold to 85. The image results were analyzed as slice in units of 35 μm. Data analysis results were presented as [adipose volume (defined by gray scale value 40-85)/average fat volume of HFD-C group]*10000.

Fat volume in the treated area was significantly reduced in the OP-A group compared independently to the HFD-C group (placebo/control) (p<0.01) and when compared to the OP-A1 (p<0.05) and OP-A2 groups (p<005). There was no statistical difference between OP-A1 or OP-A2 and TIED-C (p>0.05), even though the fat volume in the treated area was decreased in the OP-A1 and OP-A2 groups. The results obtained are provided in Table 10 below:

TABLE 10
Levels of Local Fat Reduction

| Group | HFD-C | OP-A1 | OP-A2 | OP-A |
|---|---|---|---|---|
| Fat Volume in the treated area | 100.0 ± 24.5 | 80.5 ± 42.0 | 87.0 ± 41.4 | 48.1 ± 34.4 **^{, #, $} |
| (% of HFD-C) | | | | |
| Fat Volume change in the treated are | — | −19.5 ± 42.0 | −13.0 ± 41.4 | −51.9 ± 34.4 **^{, #, $} |
| compared to HFD-C (%) | | | | |
| Fasting body weight (%) | 100.0 ± 4.0 | 96.8 ± 4.6 | 100.0 ± 7.9 | 98.0 ± 6.1 |

*p < 0.05,
** p < 0.01,
***p < 0.001 compared with HFD-C.
^{#} p < 0.05,
^{##}p < 0.01,
^{###}p < 0.001 compared with OP-A1.
^{$} p < 0.05,
^{$$}p < 0.01,
^{$$$}p < 0.001 compared with OP-A2.

### Example 3: Assessment of Stability of Exemplary Pharmaceutical Formulations

This example assesses the stability of exemplary pharmaceutical formulations disclosed herein comprising curcumin or resveratrol having a range of pH values from 4.5 to 9.0.

### A. Stability of Pharmaceutical Formulations Comprising Curcumin (A1 Group)

Pharmaceutical formulations comprising 0.4% curcumin, 16% ELP, 0.39% NaCl, 0.20 Na₂HPO₄, and 0.02% KH₂PO₄ were prepared. The pH of the formulation was adjusted to values ranging from 4.5 to 9.0 (designated as samples A1-PH-1 through A1-PH-6, respectively; see Table 11 below). Tables 12A and 12B below provide a summary of the appearance, osmolarity, pH, and stability immediately after preparation (Table 12A) and one day after preparation (Table 12B).

TABLE 11
Composition of A1 Group Pharmaceutical Formulations

| Sample | A1-PH-1 | A1-PH-2 | A1-PH-3 | A1-PH-4 | A1-PH-5 | A1-PH-6 |
|---|---|---|---|---|---|---|
| Curcumin | 0.4% | 0.4% | 0.4% | 0.4% | 0.4% | 0.4% |
| ELP | 16% | 16% | 16% | 16% | 16% | 16% |
| NaCl | 0.39% | 0.39% | 0.39% | 0.39% | 0.39% | 0.39% |
| Na₂HPO₄ | 0.2% | 0.2% | 0.2% | 0.2% | 0.2% | 0.2% |
| KH₂PO₄ | 0.02% | 0.02% | 0.02% | 0.02% | 0.02% | 0.02% |
| Water | 82.99% | 82.99% | 82.99% | 82.99% | 82.99% | 82.99% |
| Total | 100% | 100% | 100% | 100% | 100% | 100% |
| Note | Adjust the pH | Adjust the pH | Adjust the pH | Adjust the pH | Adjust the pH | Adjust the pH |
| | value to 4.5 | value to 5.5 | value to 6.5 | value to 7.5 | value to 8.5 | value to 9.0 |

TABLE 12A
Appearance, Osmolarity, and pH of A1 Formulations After Preparation (T0)

| | | | A1-PH-3 | A1-PH-4 | A1-PH-5 | |
|---|---|---|---|---|---|---|
| | A1-PH-1 | A1-PH-2 | Orange | Orange | Dark | A1-PH-6 |
| Sample | Yellow | Yellow | yellow | yellow | orange | Dark orange |
| Appearance | liquid | liquid | liquid | liquid | liquid | liquid |
| Osmolality (mOsm/kg) | 291 | 287 | 287 | 277 | 280 | 281 |
| pH value | 4.50 | 5.45 | 6.47 | 7.50 | 8.43 | 9.06 |
| Note | | | | | | After preparation, the |
| | | | | | | conc. of curcumin is low |

TABLE 12B
Appearance, Osmolarity, and pH of A1 Formulations 1 Day After Preparation

| | A1-PH-1 | | | | | |
|---|---|---|---|---|---|---|
| | Yellow | | A1-PH-3 | A1-PH-4 | A1-PH-5 | |
| | liquid and | A1-PH-2 | Orange | Orange | Dark | A1-PH-6 |
| Sample | precipitate | Yellow | yellow | yellow | orange | Dark orange |
| Appearance | particle | liquid | liquid | liquid | liquid | liquid |
| Osmolality (mOsm/kg) | 290 | 289 | 286 | 278 | 280 | 281 |
| pH value | 4.53 | 5.46 | 6.47 | 7.44 | 8.20 | 8.82 |
| Note | Precipitation | | | | | Conc. of |
| | observed | | | | | curcumin decreased. |

Formulations comprising curcumin and adjusted to pH values between 5.5 and 8.5 remained stable through the first day following preparation. In contrast, the formulation at pH 4.5 was initially stable but was instable after 1 day, and the formulation at pH 9.0 was unstable both immediately after preparation and after 1 day of storage.

### B. Stability of Pharmaceutical Formulations Comprising Resveratrol (A2 Group)

Pharmaceutical formulations comprising 0.1% resveratrol, 10% ELP, 0.58% NaCl, 0.22% Na₂HPO₄, and 0.02% KH₂PO₄ were prepared. The pH of the formulation was adjusted to values ranging from 4.5 to 9.0 (designated as samples A2-PH-1 through A2-PH-6, respectively; Table 13 below). Tables 14A and 14B below provide a summary of the appearance, osmolarity, pH, and stability immediately after preparation (Table 14A) and two days after preparation (Table 14B).

TABLE 13
Composition of A2 Group Pharmaceutical Formulations

| Sample | A2-PH-1 | A2-PH-2 | A2-PH-3 | A2-PH-4 | A2-PH-5 | A2-PH-6 |
|---|---|---|---|---|---|---|
| Resveratrol | 0.1% | 0.1% | 0.1% | 0.1% | 0.1% | 0.1% |
| ELP | 10% | 10% | 10% | 10% | 10% | 10% |
| NaCl | 0.58% | 0.58% | 0.58% | 0.58% | 0.58% | 0.58% |
| Na₂HPO₄ | 0.22% | 0.22% | 0.22% | 0.22% | 0.22% | 0.22% |
| KH₂PO₄ | 0.02% | 0.02% | 0.02% | 0.02% | 0.02% | 0.02% |
| Water | 89.08% | 89.08% | 89.08% | 89.08% | 89.08% | 89.08% |
| Total | 100% | 100% | 100% | 100% | 100% | 100% |
| Note | Adjust the pH | Adjust the pH | Adjust the pH | Adjust the pH | Adjust the pH | Adjust the pH |
| | value to 4.5 | value to 5.5 | value to 6.5 | value to 7.5 | value to 8.5 | value to 9.0 |

TABLE 14A
Appearance, Osmolarity, and pH of A2 Formulations After Preparation (T0)

| | A2-PH-1 | A2-PH-2 | A2-PH-3 | A2-PH-4 | A2-PH-5 | A2-PH-6 |
|---|---|---|---|---|---|---|
| Sample | Colorless | Colorless | Colorless | Colorless | Colorless | Colorless |
| Appearance | liquid | liquid | liquid | liquid | liquid | liquid |
| Osmolality (mOsm/kg) | 309 | 309 | 303 | 296 | 295 | 297 |
| pH value | 4.51 | 5.51 | 6.50 | 7.50 | 8.51 | 9.00 |

TABLE 14B
Appearance, Osmolarity, and pH of A2 Formulations 2 Days After Preparation

| | A2-PH-1 | A2-PH-2 | A2-PH-3 | A2-PH-4 | A2-PH-5 | A2-PH-6 |
|---|---|---|---|---|---|---|
| Sample | Colorless | Colorless | Colorless | Colorless | Light yellow | Light yellow |
| Appearance | liquid | liquid | liquid | liquid | liquid | liquid |
| Osmolality (mOsm/kg) | 310 | 307 | 303 | 293 | 292 | 294 |
| pH value | 4.56 | 5.53 | 6.51 | 7.48 | 8.22 | 8.41 |
| Note | | | | | Change a little bit | Change apparently |

Formulations comprising resveratrol and adjusted to pH values between 4.5 and 7.5 remained stable through the second day following preparation. The formulation at pH 8.5 showed a minor change in stability after 2 days, whereas the formulation at pH 9.0 showed more pronounced changes after 2 days of storage, indicating reduced stability of this formulation.

### OTHER EMBODIMENTS

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

What is claimed is:

1. A pharmaceutical composition, comprising a biologically active agent, a non-ionic surfactant hydrophilic-lipophilic balance (HLB) value ≥10, and an osmolality adjusting agent;
wherein the biologically active agent comprises a curcumin compound, a resveratrol compound, or a combination thereof,
wherein the pharmaceutical composition has an osmolality value ranging from 300±70 mOsm/kg,
optionally wherein the pharmaceutical composition has a pH value ranging from 3 to 8.5.

2. The pharmaceutical composition of claim 1, wherein the curcumin compound is curcumin; and/or wherein the resveratrol compound is resveratrol.

3. The pharmaceutical composition of claim 2, wherein the biologically active agent comprises curcumin, the concentration of which in the composition is 0.04% to 5% by weight.

4. The pharmaceutical composition of claim 3, wherein the concentration of curcumin in the composition is about 0.1% to 2.5% by weight.

5. The pharmaceutical composition of claim 3, wherein the pH of the composition ranges from about 5.5 to about 8.5.

6. The pharmaceutical composition of claim 2, wherein the biologically active agent comprises resveratrol, the concentration of which in the composition is 0.04% to 20% by weight.

7. The pharmaceutical composition of claim 6, wherein the concentration of resveratrol in the composition is about 0.05% to 5% by weight.

8. The pharmaceutical composition of claim 6, wherein the composition has a pH ranging from about 4.5 to about 8.5.

9. The pharmaceutical composition of claim 1, wherein the non-ionic surfactant comprises PEGylated oils, polyoxyethylene, polyoxyethylene castor oil derivatives, or a combination thereof.

10. The pharmaceutical composition of claim 9, wherein the non-ionic surfactant comprises polyoxyl-35 castor oil, polyoxyl 40 hydrogenated castor oil, polyoxyl 15 hydroxystearate or a combination thereof.

11. The pharmaceutical composition of claim 1, wherein the composition comprises the non-ionic surfactant at a concentration of 5% to 99% by weight.

12. The pharmaceutical composition of claim 1, wherein the osmolality adjusting agent comprises an organic agent selected from the group consisting of dextrose, mannitol, sorbitol, glycerin, and propylene glycol, an inorganic compound selected from the group consisting of sodium chloride, potassium chloride, dibasic potassium phosphate, sodium hydrogen phosphate, sodium citrate dihydrate, and sodium sulfite.

13. The pharmaceutical composition of claim 12, wherein the osmolality adjusting agent at a concentration of 0.05% to 0.9% by weight; optionally wherein the osmolality adjusting agent is sodium chloride.

14. The pharmaceutical composition of claim 1, wherein the composition has a weight ratio of the biologically active agent to non-ionic surfactant ranging from 1:5 to 1:500.

15. The pharmaceutical composition of claim 1, wherein the non-ionic surfactant and the biologically active agent form a plurality of nanoparticles.

16. The pharmaceutical composition of claim 15, wherein the nanoparticles have an average particle size of up to 200 nm and a polydispersity index (PDI) value of up to 0.4.

17. A method for reducing fat or adipocytes in a subject, comprising:
administering to a subject in need thereof an effective amount of a pharmaceutical composition set forth in claim 1.

18. A method for alleviating a condition caused by lipoma in a subject, comprising: administering to a subject in need thereof an effective amount of a pharmaceutical composition set forth in claim 1.
